(19) United States
(12) Patent Application Publication
(10) Pub. No.: US 2006/0036302 A1
(43) Pub. Date: Feb. 16, 2006

(54) Methods of inducing protective hypothermia of organs

(76) Inventors: Kenneth E. Kasza, Palos Park, IL (US); John J. Oras, Des Plaines, IL (US); David Gustav Beiser, Chicago, IL (US); HyunJin Son, Naperville, IL (US); Arieh L. Shalhav, Chicago, IL (US); Brett A. Laven, Riverwoods, IL (US); Lance B. Becker, Chicago, IL (US); Terry Vanden Hoek, Chicago, IL (US)

Correspondence Address: BRINKS HOFER GILSON & LIONE, P.O. BOX 10395, CHICAGO, IL, 60610, US

(21) Appl. No.: 11/140,500

(22) Filed: May 27, 2005

Related U.S. Application Data
(60) Provisional application No. 60/575,234, filed on May 28, 2004.
(60) Provisional application No. 60/607,340, filed on Sep. 3, 2004.

(51) Int. Cl. A61F 7/12

(52) U.S. Cl. 607/105; 128/898

(57) ABSTRACT

Methods of inducing protective hypothermia of an organ are described that include delivering a phase-change particulate slurry to at least a portion of the organ; and reducing a temperature of the organ through heat exchange with the phase-change particulate slurry. Therapeutically acceptable duration of artificially-induced ischemia of the organ is prolonged.

### RELATED APPLICATIONS

This application claims the benefit of U.S. Provisional Application No. 60/575,234, filed May 28, 2004, and U.S. Provisional Application No. 60/607,340, filed Sep. 3, 2004, the entire contents of both of which are incorporated herein by reference, except that in the event of any inconsistent disclosure or definition from the present application, the disclosure or definition herein shall be deemed to prevail.

### FEDERALLY SPONSORED RESEARCH OR DEVELOPMENT

This invention was made in part with Government support (Grant No. HL67630 awarded by the National Institutes of Health, and Contract No. W-31-109-ENG-38 between the U.S. Department of Energy and The University of Chicago representing Argonne National Laboratory). The Government may have certain rights in this invention.

### TECHNICAL FIELD

The present invention relates generally to techniques for inducing protective hypothermia of organs targeted for surgical procedures.

### BACKGROUND

When an organ is targeted for a surgical procedure—whether a minimally invasive procedure (e.g., laparoscopy, endoscopy or the like) or a conventional surgery (e.g., open-cavity procedures)—it is frequently desirable to induce a regional ischemia of the organ (e.g., through hilar or vascular clamping) in order to temporarily reduce or substantially stop the flow of biological fluids (e.g., blood) into and out of the organ, thereby enabling a surgeon to operate on the organ.

Unfortunately, prolonged periods of warm ischemia may result in serious and/or permanent damage to the organ. Since there is only a very small therapeutically acceptable window in which organ ischemia may be tolerated without resulting in concomitant damage to the organ, the surgeon is faced with increased technical and logistic challenges to successfully performing the requisite procedures in the available timeframe.

To increase the duration of therapeutically acceptable organ ischemia, techniques have been suggested for inducing protective hypothermia of the targeted organ. In conventional, open-cavity surgeries, these techniques have involved hand-packing the targeted organ with a dendritic ice slush. However, the practicality of conventional ice slush is greatly hampered by the tendency of the ice particles to agglomerate (e.g., freeze together), which renders their manipulation during surgery (e.g., to expose a portion of the organ to which access is required) difficult. In addition, conventional ice slush mixtures tend to cluster during delivery and storage, which makes it extremely difficult and impractical to deliver these materials through cannulae, tubing, syringes, and similar devices. As a result, the use of these ice slush mixtures during minimally invasive procedures (e.g., wherein delivery of the ice slush through an access port of a laparoscope or similar device would be needed) is simply not feasible.

More recently, techniques have been suggested whereby a single phase coolant (e.g., cooled saline) is perfused to an organ in order to induce hypothermia thereof. Unfortunately, because of the low cooling capacity of such single phase coolants, the reductions in organ temperature achievable typically are not competitive with the cooling efficiency of conventional ice slush techniques.

Thus, the need persists for a method of inducing protective hypothermia of an organ that avoids the drawbacks described above, and which is suitable for use in minimally invasive procedures.

### SUMMARY

The scope of the present invention is defined solely by the appended claims, and is not affected to any degree by the statements within this summary.

In some embodiments, a method of inducing protective hypothermia of an organ includes: (a) delivering a phase-change particulate slurry to at least a portion of the organ; and (b) reducing a temperature of the organ through heat exchange with the phase-change particulate slurry. A therapeutically acceptable duration of artificially-induced ischemia of the organ is thereby prolonged.

In some embodiments, a method of inducing protective hypothermia of an organ targeted for a surgical procedure includes: (a) delivering a phase-change particulate slurry to at least a portion of the organ; (b) reducing an initial temperature of the organ to about 15 degrees Celsius or lower through heat exchange with the phase-change particulate slurry; (c) inducing ischemia of the organ; and (d) performing a surgical procedure on the organ. The phase-change particulate slurry contains an ice slurry containing substantially globular ice particles suspended in a carrier comprising water. The ice particles have an average diameter of about 100 micrometers or less, and comprise at least about 30 percent by weight of the phase-change particulate slurry.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a phase-change particulate slurry embodying features of the present invention (left, top and bottom), and a conventional dendritic ice slush (right, top and bottom).

FIG. 2 shows a laparoscopic procedure and laparoscopic surgery entry ports for surgical tools, endoscope, and slurry delivery tube.

FIG. 3 shows an endoscope monitor view of procedures during minimally invasive laparoscopic surgery.

FIG. 4 shows a slurry delivery 12-inch long tube for use in accordance with the present invention, which is shown inserted through a commercial laparoscopic port and interfaced with a slurry delivery pump tube.

FIG. 5 shows an excised swine kidney of nominally 100 g, which is instrumented with thermocouples during conditioning in a heated saline water tempering bath (37° C.) prior to cooling.

FIG. 6 shows a blender for making phase-change particulate slurry for use in accordance with the present invention.

FIG. 7 shows a slurry preconditioning mixing apparatus and pumping system for delivering phase-change particulate slurry to an organ in accordance with the present invention.

FIG. 8 shows a calorimeter apparatus for measuring actual cooling capacity of phase-change particulate slurry just prior to delivery.

FIG. 9 shows an insulated box containing an excised swine kidney that is nearly completely covered with an ice slurry, as well as temperature recorders and thermocouples inserted into the kidney to measure cooling.

FIG. 10 shows a warm-wall simulator including a jacketed double-walled water tempering beaker fed by a thermo-regulated recirculation system, wherein a swine kidney is shown resting on thin plastic sheet representing normal body temperature tissue.

FIG. 11 shows a thermo-regulated recirculation system feeding 37° C. water to the warm-wall simulator tempering beaker of FIG. 10.

FIG. 12 shows a swine kidney placed on top of the warm-wall simulator plastic sheet shown in FIG. 10 and partially covered with ice slurry.

FIG. 13 shows a plot of kidney ice slurry cooling temperature versus time for complete covering of a first swine kidney with slurry.

FIG. 14 shows a plot of kidney ice slurry cooling temperature versus time for partial covering of a second swine kidney in the warm-wall simulator of FIG. 10.

FIG. 15 shows a plot of kidney ice slurry cooling temperature versus time for internal cooling of a third swine kidney on an insulated surface with thermocouple no. 6 on top.

FIG. 16 shows a plot of kidney ice slurry cooling temperature versus time for internal cooling of the third swine kidney in the warm-wall simulator of FIG. 10 with thermocouple no. 6 on top.

FIG. 17 shows a plot of kidney ice slurry cooling temperature versus time for internal cooling of a fourth swine kidney in the warm-wall simulator of FIG. 10 with thermocouple no. 6 on top.

FIG. 18 shows a plot of kidney ice slurry cooling temperature versus time for internal cooling of the fourth swine kidney on an insulated surface with thermocouple no. 6 on top.

FIG. 19 shows an endoscope view of a first swine kidney prior to placement of thermocouples in the kidney and delivery of ice slurry.

FIG. 20 shows an external view of the subject swine, with 8 hypodermic needle access ports for routing thermocouples into the kidney shown in the foreground, and a white insulated slurry delivery tube conveying slurry into the special SS injector tube interfaced with a standard laparoscopic port shown in the background.

FIG. 21 shows an endoscope view of the swine kidney of FIG. 19 nearly completely covered with slurry, as well as the actual SS delivery tube tip depositing the slurry; also shown, in the upper left, are 4 of the 8 thermocouple leads associated with the thermocouples imbedded in the kidney.

FIG. 22 shows a plot of ice slurry cooling kidney temperature versus time for minimally invasive laparoscopic transperitoneal access surgery.

FIG. 23 shows ice slurry kidney cooling via open-cavity retroperitoneal access through a flank incision for thermocouple placement and slurry pumped delivery by direct visual viewing.

FIG. 24 shows a plot of kidney ice slurry cooling temperature versus time for conventional open-cavity surgery using retroperitoneal access through a flank incision.

### DETAILED DESCRIPTION

Highly efficient and convenient methods for inducing protective hypothermia of organs through the use of phase-change particulate slurries have been discovered and are described hereinbelow. Experiments have shown that these methods are applicable in both minimally invasive surgeries (e.g., laparoscopic procedures) as well as in conventional open-cavity surgeries, and that they provide organ temperature reductions sufficient for preventing ischemia damage during surgical procedures.

By way of introduction, a method of inducing protective hypothermia of an organ embodying features of the present invention includes (a) delivering a phase-change particulate slurry to at least a portion of the organ; and (b) reducing a temperature of the organ through heat exchange with the phase-change particulate slurry, such that a therapeutically protective level of hypothermia is induced producing a therapeutically acceptable duration of artificially-induced ischemia of the organ.

As used herein, the phrase “therapeutically protective level of hypothermia” refers to a degree of cooling of an organ that is generally sufficient to decrease, minimize, and/or substantially prevent damage to the organ caused during a surgical procedure, such as the ischemia damage that may otherwise result from subjecting the organ to clamp-off procedures in the absence of protective cooling.

As used herein, the phrase “therapeutically acceptable duration” as used in reference to artificially-induced ischemia refers to a period of time beyond which the risk of ischemia-induced damage would outweigh the surgical benefit of inducing organ ischemia.

As used herein, the phrase “artificially-induced” as used in reference to ischemia refers to a deliberate action (e.g., taken on the part of a surgeon) to cut off and/or restrict the flow of biological fluids into and/or out of an organ. As such, this phrase is distinguished from a pre-existing or “naturally-occurring” ischemia, such as a blockage or obstruction of a vein, artery, duct or the like.

As used herein, the phrase “phase-change particulate slurry” refers to a slurry in which small particles of a phase-change material are suspended in a carrier fluid. In some embodiments, the phase-change material is ice and the carrier fluid is liquid water. In other embodiments, the carrier fluid is saline solution (e.g., an aqueous solution of sodium chloride), a perflourocarbon, or a combination thereof. Phase-change particulate slurries for use in accordance with the present invention are further described in U.S. Pat. No. 6,244,052 B1; U.S. Pat. No. 6,413,444 B1; U.S. Pat. No. 6,547,811 B1; United States Patent Publication No. U.S. 2003/0066304A1; and United States Patent Publication No. U.S. 2004/0187512 A9, all of which are assigned to the assignee of the present invention. The entire contents of all five of these documents are hereby incorporated herein by reference, except that in the event of any inconsistent disclosure or definition from the present application, the disclosure or definition herein shall be deemed to prevail.

Phase-change particulate slurries used in accordance with the present invention are engineered to have high cooling capacity, fluidity, and particle loading. In some embodiments, as further described below, the phase-change particulate slurry comprises an ice slurry. As used herein, the phrase “ice slurry” refers to slurries comprising ice particles suspended in a carrier comprising water. In some embodiments, the carrier further comprises sodium chloride.

In some embodiments, the ice particles and/or the water carrier optionally contains one or more chemical additives (e.g., freezing point depressants, including but not limited to sodium chloride and the like; pharmaceutical agents; and all manner of biocompatible agents capable of providing a therapeutically beneficial effect to the organ and/or patient, and/or the introduction of which is desirable in connection with a surgical procedure to be performed). For embodiments in which the phase-change particulate slurry contains sodium chloride, it is desirable that the saline concentration of the phase-change particulate slurry be chosen so as to be biocompatible with the patient, such that chemical imbalances triggered by saline from the phase-change particulate slurry may be avoided. In some embodiments, the saline concentration is between about 0.5 and about 3.0 percent by weight.

In some embodiments, all ingredients of the phase-change particulate slurry (e.g., ice particles, carrier water, dissolved sodium chloride and/or other chemical additives, and the like) are sterile, medical-grade materials substantially free from microorganisms (e.g., bacteria, viruses, and the like) dirt, impurities, etc.

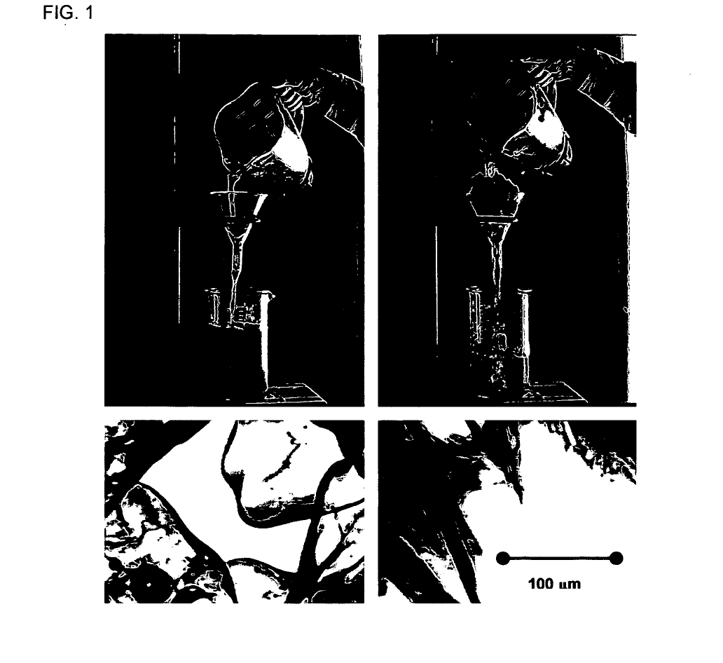

In some embodiments, as shown in FIG. 1 (left, top and bottom), an ice slurry in accordance with the present invention comprises substantially smooth and/or substantially globular ice particles with an average diameter of about 100 μm or less, such that the slurry exhibits superior fluid dynamic properties rendering it suitable for pumping through intravenous catheters, hypodermic needles, cannulae, medical delivery tubing, and the like. By contrast, conventional dendritic ice (FIG. 1, right, top and bottom), as produced by conventional slush machines, cannot be readily pumped and produces plugging when poured.

Phase-change particulate slurries in accordance with the present invention, which in some embodiments comprise ice slurries, may be engineered to have very high particle loading and, therefore, significantly higher cooling capacity than a non-phase-change single-phase coolant such as cooled saline. In some embodiments, the phase-change material (e.g., ice particles) comprises at least about 20 percent by weight of the phase-change particulate slurry (e.g., ice slurry). In some embodiments, the phase-change material comprises at least about 30 percent by weight of the phase-change particulate slurry. In some embodiments, the phase-change material comprises at least about 40 percent by weight of the phase-change particulate slurry. In some embodiments, the phase-change material comprises at least about 50 percent by weight of the phase-change particulate slurry. In some embodiments, the weight of the phase-change material approaches and/or exceeds about 60 percent by weight of the phase-change particulate slurry.

Moreover, the phase-change particulate slurries also exhibit very high fluidity, storability, and freedom from plugging in small delivery tubes, as shown in FIG. 1, such that the slurries may be easily pumped or otherwise delivered to a cooling target (e.g., an organ targeted for surgery) in minimally invasive as well as in open-cavity conventional surgeries. In some embodiments, the phase-change particulate slurry is delivered to an organ—either externally or internally, as further described below—at a rate of at least about 100 milliliters per minute. In some embodiments, the phase-change particulate slurry is delivered to the organ at a rate of at least about 150 milliliters per minute. In some embodiments, the phase-change particulate slurry is delivered to the organ at a rate of at least about 175 milliliters per minute. In some embodiments, the phase-change particulate slurry is delivered to the organ at a rate of at least about 200 milliliters per minute.

In some embodiments, the phase-change particulate slurries for use in accordance with the present invention can be delivered to an organ through a variety of small diameter tubes. Indeed, phase-change particulate slurries used in accordance with the present invention have been demonstrated to be pumpable through delivery channels as small as a 14 gauge hypodermic needle, although pumping through even smaller diameter channels may be possible as well. Materials and devices for the delivery of phase-change particulate slurries in accordance with the present invention are further described in co-pending U.S. patent application Ser. No. 11/038,570, filed Jan. 18, 2005, and assigned to the assignee of the present invention. The entire contents of this document are hereby incorporated herein by reference, except that in the event of any inconsistent disclosure or definition from the present application, the disclosure or definition herein shall be deemed to prevail. The use of phase-change particulate slurries in a variety of other medical devices, and devices for delivering phase-change particulate slurries over a wide range of applications are further described in the U.S. Patents, Patent Publications, and Patent Application incorporated by reference hereinabove.

In some embodiments, methods of inducing protective hypothermia of an organ embodying features of the present invention further include performing a surgical procedure on the organ. As used herein, the phrase “surgical procedure” is to be understood in a very broad sense as including all manner of disease diagnosis and disease treatment, including but not limited to: partial organ removal (e.g., resection and/or biopsy of tumors and/or other masses); total organ removal (e.g., organ harvesting, total nephrectomy, appendectomy, and the like); partial and/or total organ replacement (e.g., artificial and/or natural organ transplants, and the like); organ preservation; and combinations thereof.

In some embodiments, the surgical procedure is initiated after the temperature of the target organ has been reduced (e.g., to or below a predetermined temperature at which the potential for ischemia-related damage is minimized and/or prevented). In some embodiments, the temperature of the organ is reduced by at least about 10 degrees Celsius. In some embodiments, the temperature of the organ is reduced by at least about 15 degrees Celsius. In some embodiments, the temperature of the organ is reduced by at least about 20 degrees Celsius. In some embodiments, the temperature of the organ is reduced by at least about 25 degrees Celsius.

Methods of inducing protective hypothermia of an organ embodying features of the present invention may be used in human medicine and all manner of veterinary medicine, including but not limited to the treatment of humans and wild and domestic animals (e.g., pigs, dogs, cats, horses, gorillas, etc.), birds, reptiles, etc.

In some embodiments, the patient to be treated is a mammal. In some embodiments, the patient has a normal body temperature of about 37 degrees Celsius (e.g., humans, swine, etc.), such that an initial temperature of the organ targeted for surgical procedure is about 37 degrees Celsius. In some embodiments, the initial temperature of 37 degrees Celsius is reduced to about 15 degrees Celsius or lower. In some embodiments, the initial temperature is reduced to about 15 degrees Celsius or lower in less than about 20 minutes. In some embodiments, the initial temperature is reduced to about 15 degrees Celsius or lower in less than about 15 minutes. In some embodiments, the initial temperature is reduced to about 15 degrees Celsius or lower in less than about 10 minutes.

In some embodiments, the surgical procedure comprises a minimally invasive procedure, including but not limited to: laparoscopic procedures, laparoscopically-assisted procedures (i.e., operative procedures performed using a combination of laparoscopic and conventional open-cavity techniques), thoracoscopic procedures, endoscopic procedures, and combinations thereof.

In some embodiments, the minimally invasive procedure comprises a laparoscopic technique. In such embodiments, the phase-change particulate slurry may be delivered to an organ through an access port in a laparoscope. In other embodiments, the surgical procedure comprises an open-cavity procedure.

In some embodiments, at least a portion of an exterior surface of the organ is coated with the phase-change particulate slurry (e.g., external cooling, further described below). In such embodiments, methods embodying features of the present invention may further include sculpting a coating of the deposited phase-change particulate slurry (e.g., using the tip of the delivery device that was used to deliver the phase-change particulate slurry; a Kitner dissector; or the like). In some embodiments, methods embodying features of the present invention further include removing at least a portion of the phase-change particulate slurry from the exterior surface of the organ after the temperature of the organ is reduced to a desired value (e.g., a predetermined temperature at or below which ischemia damage to an organ is minimized and/or prevented).

In some embodiments, at least a portion of the phase-change particulate slurry is delivered to an interior surface of the organ (e.g., internal cooling, further described below). In such embodiments, the phase-change particulate slurry may be delivered through a biological channel coupled to the interior surface. As used herein, the term “coupled” is intended broadly to encompass both direct and indirect coupling. Thus, a biological channel and an organ are said to be coupled together when they are directly connected (e.g. by direct contact) and/or functionally engaged, as well as when the biological channel is functionally engaged with an intermediate part which is functionally engaged either directly or via one or more additional intermediate parts with the organ. Also, a biological channel and an organ are said to be coupled when they are functionally engaged (directly or indirectly) at some times and not functionally engaged at other times. In some embodiments, the biological channel includes but is not limited to: hila; veins; arteries; and the like; and combinations thereof. In some embodiments in which phase-change particulate slurry is delivered to an interior of an organ, the organ is at least partially collapsed prior to delivery of the phase-change particulate slurry (e.g., such that the organ is increased and/or expanded by the phase-change particulate slurry).

In some embodiments, at least a portion of the phase-change particulate slurry is delivered to an exterior surface of the organ and at least a portion of the phase-change particulate slurry is delivered to an interior surface of the organ (i.e., a combination of internal and external cooling).

In some embodiments, methods of inducing protective hypothermia of an organ embodying features of the present invention further include inducing ischemia of the organ targeted for surgical procedure and/or any additional organs for which temporary ischemia is advisable in order to maximize the success of the surgical procedure. In some embodiments, the ischemia is induced via full hilar clamping. In some embodiments, the ischemia is induced via partial hilar clamping.

The type of organ for which protective hypothermia may be induced in accordance with the present invention is not restricted and includes but is not limited to: kidneys; liver; pancreas; heart; brain; appendix; spleen; colon; lungs; bladder; prostate; stomach; and the like; and combinations thereof.

In some embodiments, further described below, the organ comprises a kidney, and the methods embodying features of the present invention include performing a surgical procedure on the kidney. In some embodiments, the surgical procedure comprises a full or partial nephrectomy. In some embodiments, the nephrectomy is performed using a minimally invasive procedure, including but not limited to a laparoscopic technique. In such embodiments, the phase-change particulate slurry may be delivered to the kidney (externally and/or internally) through an access port in a laparoscope. In other embodiments, the nephrectomy is performed using an open-cavity procedure.

In some embodiments, at least a portion of an exterior surface of the kidney is coated with the phase-change particulate slurry (e.g., external cooling). The experiments described below demonstrate that an ice slurry may be used to successfully coat a kidney, and that the ice slurry adheres well to the serosal surface of the kidney.

In other embodiments, at least a portion of the phase-change particulate slurry is delivered to an interior surface of the kidney (e.g., internal cooling). In such embodiments, the phase-change particulate slurry may be delivered to the interior of the kidney through a ureter thereof. In other embodiments, the phase-change particulate slurry is delivered to both an exterior surface and an interior surface of the kidney (e.g., a combination of external and internal cooling).

Representative applications and additional examples of methods embodying features of the present invention are further described below.

In previous work, described for example in the U.S. Patents, Patent Publications, and Patent Application incorporated by reference hereinabove, phase-change particulate slurries have been shown to provide improved methods for quickly cooling the heart and brain to induce cell protective hypothermia during medical emergencies that tend to cause ischemia induced damage (e.g., cardiac arrest, stroke, and the like). The ability of heart and brain cells to survive severe oxygen deprivation may be dramatically enhanced by rapid cooling (e.g., cell protective hypothermia). In this previously described research, the ice slurry may be delivered to the lungs, stomach, or carotid artery (which are used as in-body heat exchangers), or by direct injection into the femoral vein. The slurry-cooled blood is then circulated to the brain/heart cooling targets by chest compressions to induce blood flow for cardiac arrest, and by functioning cardiac output for stroke patients to achieve rapid cooling. The resulting cooling reduces further ischemic injury yielding additional time for treatment.

The use of our phase-change particulate slurries, delivery approaches and delivery devices to cool other organs in addition to the brain and heart (e.g., the kidneys, liver, pancreas, etc.) have now been shown to provide ischemia protection during minimally invasive surgery and conventional open-cavity surgery. These newly discovered methods may be employed in a wide variety of surgical procedures, including but not limited to organ harvesting, organ transplants, organ preservation, and the like, and combinations thereof. Additional medical applications include but are no limited to: cardiac arrest; myocardial infarctions; stroke; severe head trauma; severe blood loss; fever control; heat stroke; sports drink; and the like; and combinations thereof.

In additional embodiments, such as those described in the representative examples provided below, the use of phase-change particulate slurries has been successfully extended to applications in urology surgery in which organ tissue damage caused by ischemia represents a significant problem. Moreover, this problem will only be further aggrandized by the current trend to use minimally invasive surgery.

In the representative examples described below, a series of ice slurry cooling experiments involving eight kidneys removed from large swine are described. Additional experiments on a large swine with intact kidneys using laparoscopic surgical procedures similar to those used on humans are also described, in which, for the first time, ice slurry was successfully delivered to coat and cool kidneys guided by endoscopic viewing.

One cooling method to reduce ischemia damage currently under development for use by others in kidney surgery involves the use of a saline single phase coolant for retrograde intra-renal cooling via ureteral access (Landman et al., “Renal Hypothermia Achieved by Retrograde Intracavity Saline Perfusion,” Journal of Endourology 2002, 16, 445). A second method involves the topical application of ice slush loaded into a bag surrounding the kidney (Gill et al., “Laparoscopic Ice Slush Renal Hypothermia for Partial Nephrectomy: The Initial Experience,” Journal of Urology, 2003, 170, 52). However, there are distinct limitations to the use of both techniques as cooling methods during laparoscopic partial nephrectomy.

The topical ice slush technique involves conventional ice slush injected through sequential modified 30 milliliter syringes in the neck of a pre-positioned Endocatch bag exteriorized at a 12 mm port site. This technique achieves nadir renal parenchymal temperatures ranging from 5 to 19° C. and closely approximates the established techniques of open partial nephrectomy. However, it is very cumbersome and time consuming, requiring removal and changing of ports, enlargement of port-site incisions, and incision and mobilization of the Endocatch bag to allow tumor resection. In addition, space limitations prevent the use of this technique in retroperitoneoscopic partial nephrectomy. Moreover, the physical size of the bag may compromise operative exposure, access to the hilum, and in one case, interfered with the hilar Satinsky clamp leading to inadequate control of the renal vessels and additional blood loss.

The technique utilizing retrograde perfusion of cold saline into the renal pelvis through a pre-placed ureteral access catheter has been investigated, but nadir parenchymal temperatures of 21-24° C. do not approximate the cooling efficiency of topical ice-slush techniques. Furthermore, in the event of collecting system violation during resection, the spillage of irrigant compromises cooling efficiency and visualization. An additional limitation is the potential for ureteral injury during ureteral access sheath placement, manipulation, or during patient repositioning. Moreover, intracavitary cooling also adds costs and increases operative time as a result of the access sheath placement.

The cooling methods embodying features of the present invention, which utilize a highly loaded and high fluidity phase-change particulate slurry, may be used to great advantage over both the current method employed for open cavity surgeries, which involves the packing of the exposed organ in ice, and the above-described procedures currently under development for use in minimally invasive surgeries.

Because of the high energy content of the ice slurries used in accordance with the present invention—which is due to the phase change (e.g., melting) of the ice particles under a cooling load—the cooling content of a phase-change particulate slurry is many times greater than that of chilled water. Studies by the present inventors have shown that the slurry is preferably engineered to have appropriate ice particle characteristics, such as globularity and smoothness, as further described in the U.S. Patents, Patent Publications, and Patent Application incorporated by reference hereinabove. In some embodiments, the ice particle slurries may optionally be engineered with additional chemicals that are compatible with human tissue. The engineered ice slurries for use in accordance with the present invention are believed to have the highest ice particle loading ever achieved (in some embodiments approaching 60%). Hence, these ice slurries have a much greater cooling capacity than a non-phase-change single-phase coolant such as cooled saline. Moreover, the ice slurries also have very high fluidity, storability and freedom from plugging in very small delivery tubes; as such, they may be readily pumped through small delivery tubing to a cooling target and are ideal for use in cooling a wide range of organs during minimally invasive laparoscopic surgery.

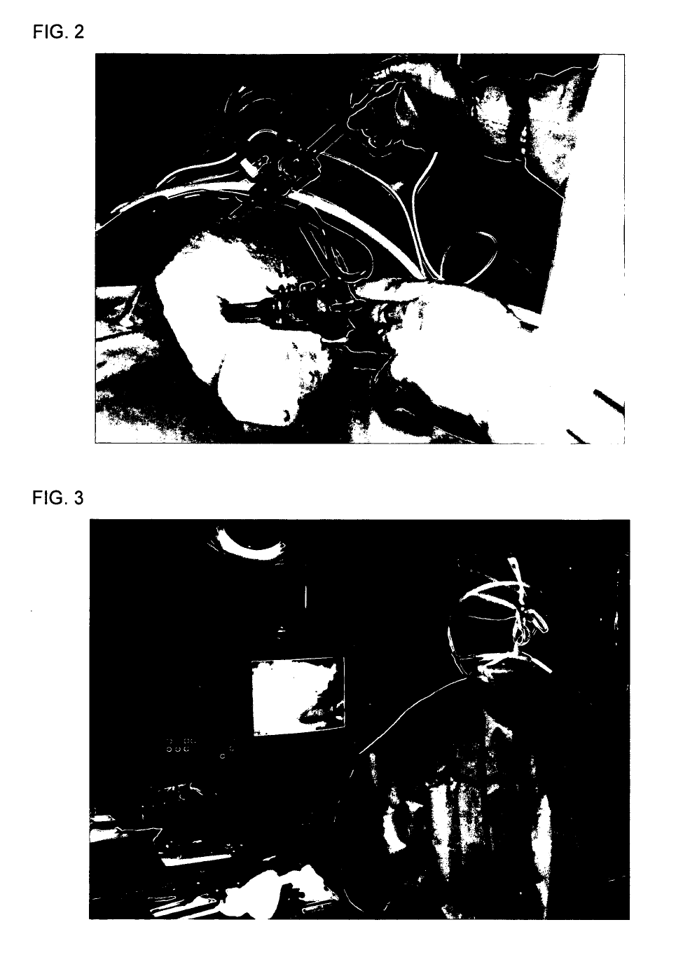

As shown in FIGS. 2-4, the ice slurry can be delivered through a variety of small diameter tubes inserted through a small access hole associated with laparoscopic surgery ports with the slurry delivery tube guided by endoscope viewing for depositing (e.g., coating) slurry around the exterior of an organ to induce protective hypothermia. In addition, the slurry can also be introduced into the organ by using a modified catheter introducer or similar small bore tube to deliver slurry directly via a blood vessel or other biological channel route (e.g., the urethra) into the organ for cooling.

If a sub-sector of an organ needs to be left uncovered from slurry to facilitate surgical tool access or visual observation, the slurry delivery tube can be positioned to accomplish this; alternatively, or in addition, the slurry can be moved around with the delivery tube to uncover the location of needed access. The slurry ice particles do not have a tendency to freeze together or cluster, thus facilitating sculpting of the coating as desired. The above-described conventional cooling method, whereby a bag containing coolant is placed around an organ and then partially removed to facilitate surgical access, is very cumbersome compared to the use of slurries in accordance with the present invention. Moreover, the conventional method will provide much smaller cooling rates because of a lower heat transfer coefficient between the conventional ice packing and the outer surface of the target organ.

In some embodiments, phase-change particulate slurry in accordance with the present invention may be delivered internally to an organ or in combination with external cooling. The internal delivery of slurry is more complex than external delivery due in part to the need to achieve correct over-pressure. Nonetheless, internal delivery may be accomplished, for example, by using a feed back control loop between the organ pressure (e.g., as measured by a transducer) and the pumped slurry delivery system to avoid possible organ over-pressurization and tearing. Based on the experimental data described below, external cooling of an organ by coating the outer surface thereof is generally sufficient for many applications. Moreover, the delivery system complexity and coolant sterility requirements for this external delivery approach are lower than for corresponding internal delivery, and are perhaps more directly compatible with current laparoscopic equipment and procedures.

Furthermore, because the highly loaded phase-change particulate slurries have such high cooling capacity, a desired cool-down is achievable with volumes which will not overload the organs, blood vessels, or cavity spaces associated with a given cooling application. In addition, the slurry may be made sterile and with a saline concentration similar to that of drip bag saline, such that when melted, chemical imbalances triggered by saline from the slurry should not be a concern.

By using methods embodying features of the present invention, rapid induction of protective hypothermia is feasible in a hospital environment and in an out-of-hospital environment without requiring the use of complex time-consuming bypass external heat exchangers; rather, the methods in accordance with the present invention may utilize in-body biological heat exchangers cooled with phase-change particulate slurry. In contrast to the fast and deep cooling achievable in accordance with the present invention, modeling and analysis of brain and heart cooling have shown that external cooling of the body cannot achieve the desired fast rate of cooling. Although conventional cooling involving the use of complex bypass heat exchanger methods may achieve rapid cooling once the patient is hooked-up, the bypass system hook-up is very time-consuming and invasive and thus is impractical in the out-of-hospital environment. In addition, the use of external body cooling devices such as ice packs, cooling caps or jackets, and fan-induced cooling are generally very slow achieving cooling rates less than 0.03° C./min, which is far too slow for many medical applications. External cooling devices also tend to cause excessive harmful patient shivering.

The following representative procedures and examples are provided solely by way of illustration, and are not intended to limit the scope of the appended claims or their equivalents.

### EXAMPLES

Ice Slurry Cooling Experiments on a Removed Kidney

The data described below demonstrates that methods embodying features of the present invention provide a way to rapidly and deeply cool an organ during minimally invasive surgery using simple slurry delivery methods, which are compatible with existing surgical laparoscopic procedures and endoscope viewing. The ice slurry is delivered by a pumping delivery system through a specially designed tube that is inserted through existing surgical ports used in minimally invasive surgery. The slurry delivery, aided by the use of endoscope video viewing, is distributed around an organ (in these representative examples, the organ is a kidney), thereby coating it. The slurry coating rapidly cools the kidney prior to clamping off of the arterial blood supply and operating on the kidney.

A series of ice slurry cooling experiments involving eight kidneys removed from large (40 kg) swine were conducted over four days. FIG. 5 shows a typical removed kidney weighting nominally 100 g and instrumented with thermocouples inserted 5 mm deep into kidney tissue prior to cooling.

Two types of experiments were performed: (a) external cooling with slurry either partially or fully covering the kidney; and (b) internal cooling using urethra delivery of coolant consisting of either ice slurry or cold saline. All methods provided substantial kidney cooling of 15 to more than 20° C. below normal temperature of 37° C. in 8 to 30 minutes. However, external cooling by coating the kidney partially or fully with slurry is the more rapid and easier to implement of the two approaches.

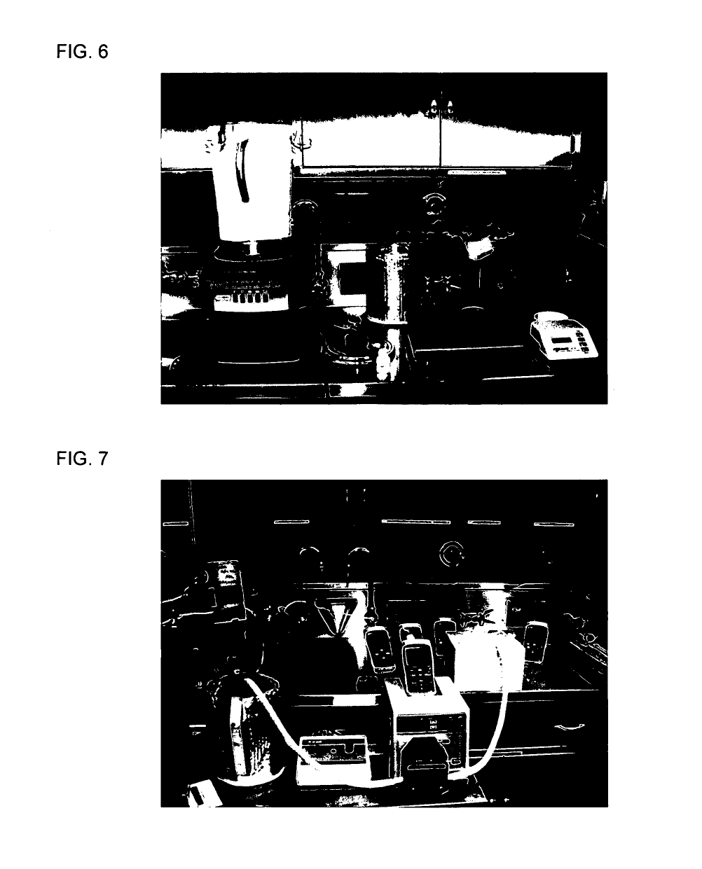

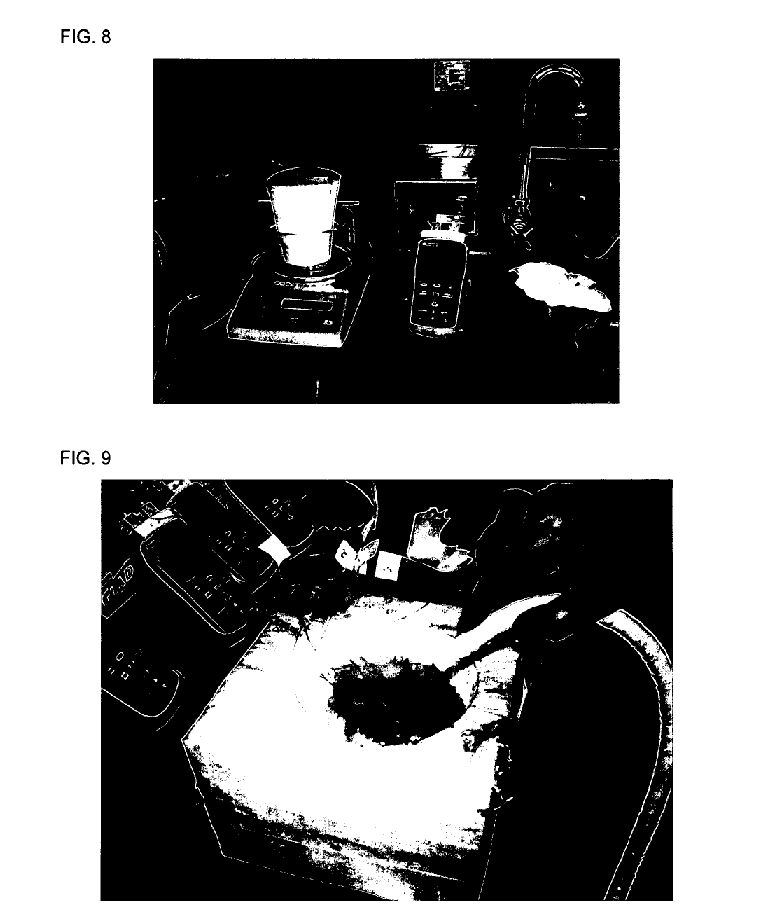

FIG. 6 shows a high-capacity blender (e.g., WARING Heavy Duty Laboratory Blender, 3 Hp) that may be used for making an ice slurry in accordance with the present invention. FIG. 7 shows a slurry preconditioning mixing apparatus and pump delivery system for supplying slurry to a kidney. Also shown in FIG. 7 are the thermocouple data recorders and an insulated Styrofoam box for cooling the removed kidney by complete immersion in slurry. FIG. 8 shows a calorimeter apparatus used for measuring actual cooling capacity of the ice slurry just prior to delivery.

Two modes of external cooling of the kidney were tested. The first approach involved complete immersion using the insulated box shown in FIGS. 7 and 9, where the kidney is shown being covered by slurry after an initial layer of slurry was deposited on the bottom of the box by pumped delivery through a slurry tube. As shown in FIG. 9, the kidney is nearly completely covered with slurry.

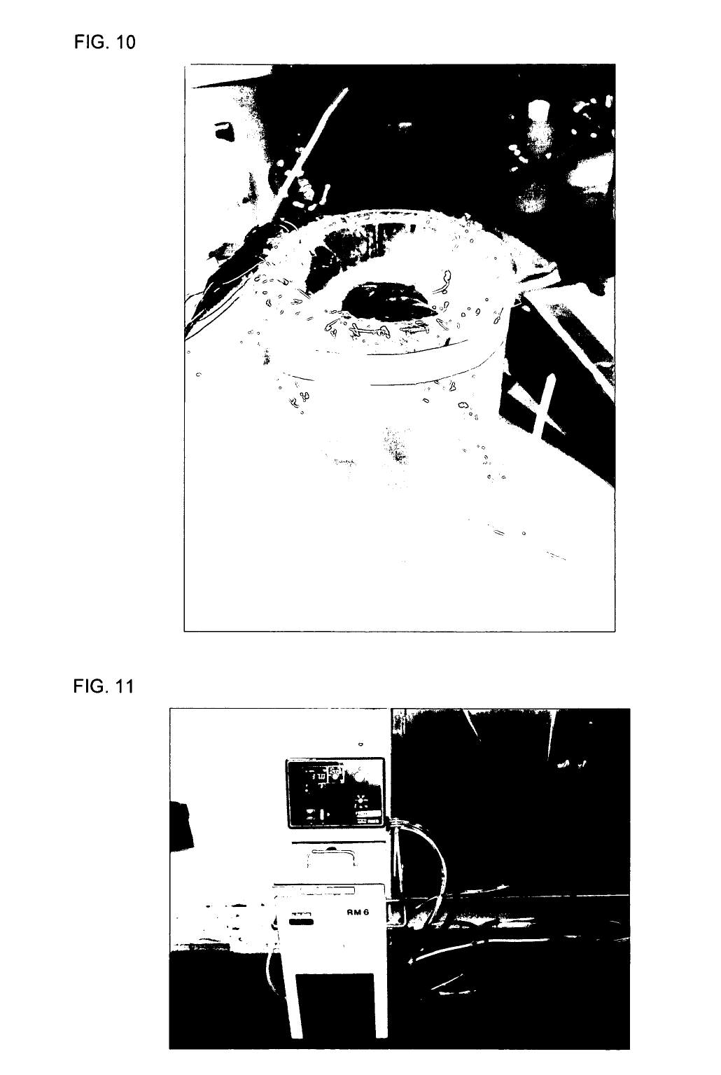

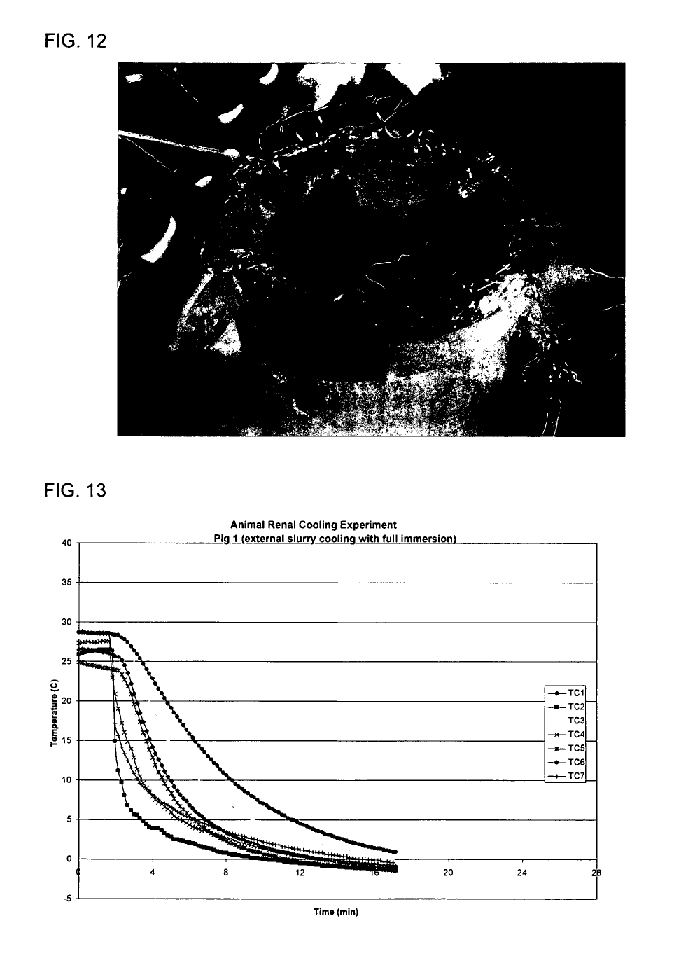

The second cooling mode involved using a warm-wall simulator to represent a less-optimum slurry cooling scenario, in which only approximately half of the kidney was covered with slurry and the other half is simulated in contact with tissue at normal body temperature. The warm wall simulator, shown in FIG. 10, includes a water-jacketed double-walled tempering beaker fed by a thermo-regulated recirculation system, shown in FIG. 11. The tempering beaker was filled with saline and covered by a thin plastic sheet to simulate contact with warm tissue. The kidney was placed on top of the sheet and then partially covered with slurry, as shown in FIG. 12.

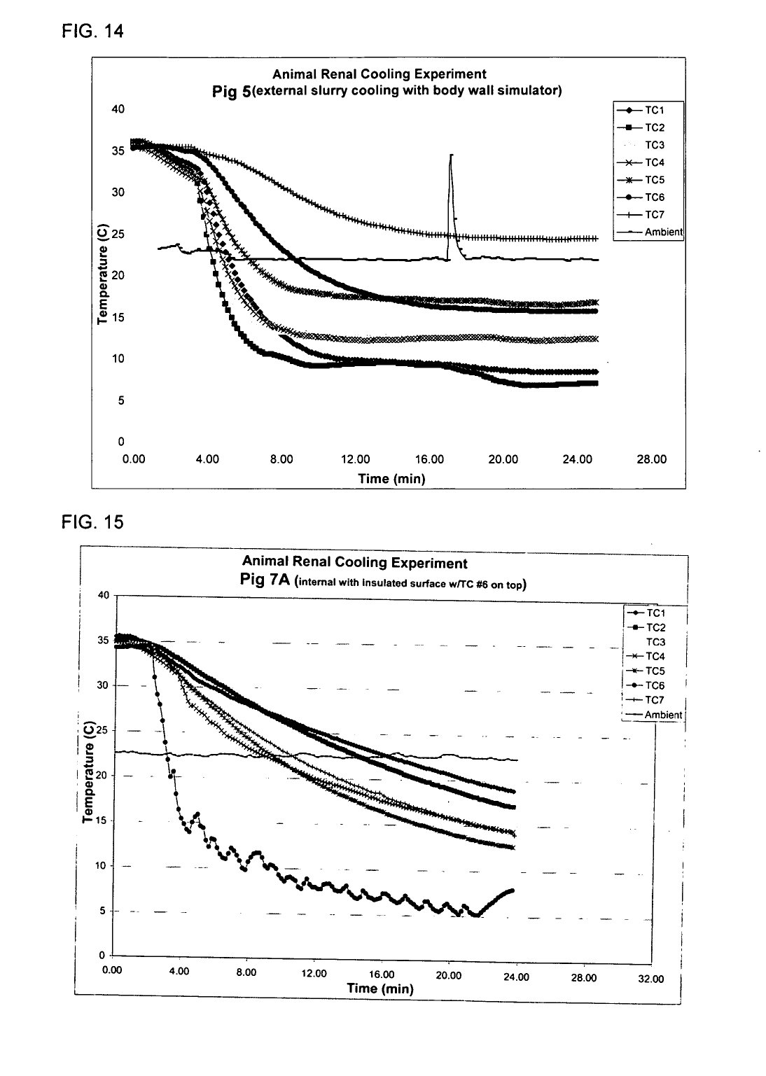

FIGS. 13 and 14 show kidney cooling data for complete covering of the kidney with slurry and partial covering in the warm-wall simulator, respectively. Both kidneys, of approximately the same mass, cooled more than 15° C. over their entire extent in less than 8 minutes after the slurry was applied. Actual slurry delivered for these two tests was less than 1 liter. Clearly, even if only ½ of the kidney is covered with slurry and the other half is in contact with warm tissue (37° C.) the cooling is very fast and deep, as shown by FIG. 14. For the fully covered kidney, a 23° C. cool down occurred in 4 minutes for a nominal cooling rate of about −5.7° C./min.

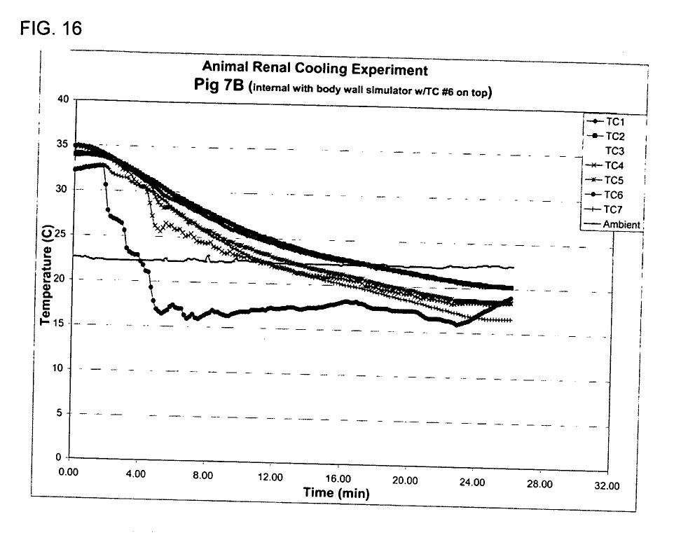

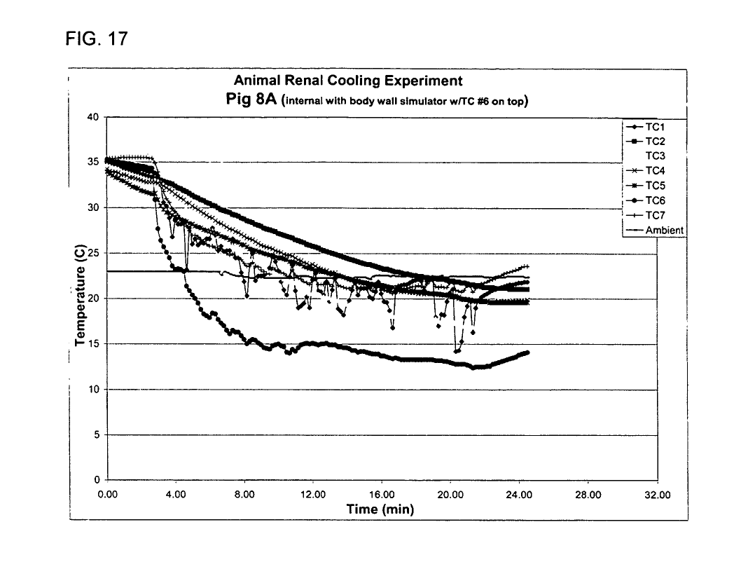

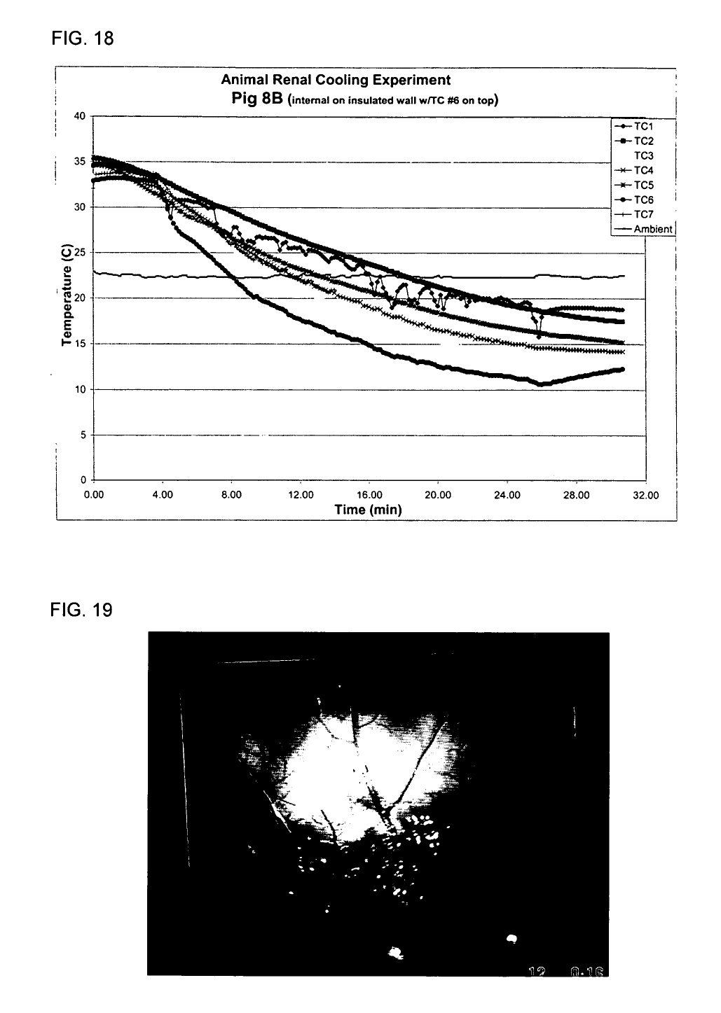

FIGS. 15-18 show additional kidney cooling data obtained in this first series of tests using internal delivery of slurry to the removed kidney. Fluctuations and/or sudden spikes in the curves shown in several of these figures are the result of slippage, detachment, and/or repositioning of the associated thermocouples during the course of the experiments. In some cases, the thermocouples may have directly contacted ice particles resulting in sudden drops in recorded temperature. In all cases, the kidneys were rapidly and deeply cooled.

This first series of tests did not involve intact perfused kidneys with metabolic heating, both of which can be heat sources in targeted organ cooling depending on surgical arterial clamp-off procedures. However, because the ice slurry has such a high cooling capacity and the heat transfer coefficient between slurry and kidney outer surface is very large, cooling of an intact kidney would also be very rapid and deep, as confirmed by the experiments described below.

In conclusion, the cooling data presented in FIGS. 13 and 14 for external cooling and FIGS. 15-18 for internal delivery demonstrates the feasibility of using our engineered phase-change particulate slurries to perform minimally invasive laparoscopic kidney or other organ cooling for ischemia protection. The experiments described below provide experimental proof of the workability and effectiveness of using ice slurry cooling to prevent ischemia damage during minimally invasive surgery on an intact organ (e.g., a kidney).

Ice Slurry Kidney Cooling of Intact Kidney Using Minimally Invasive Laparoscopic Surgical Procedures and Conventional Open-Cavity Surgical Procedures

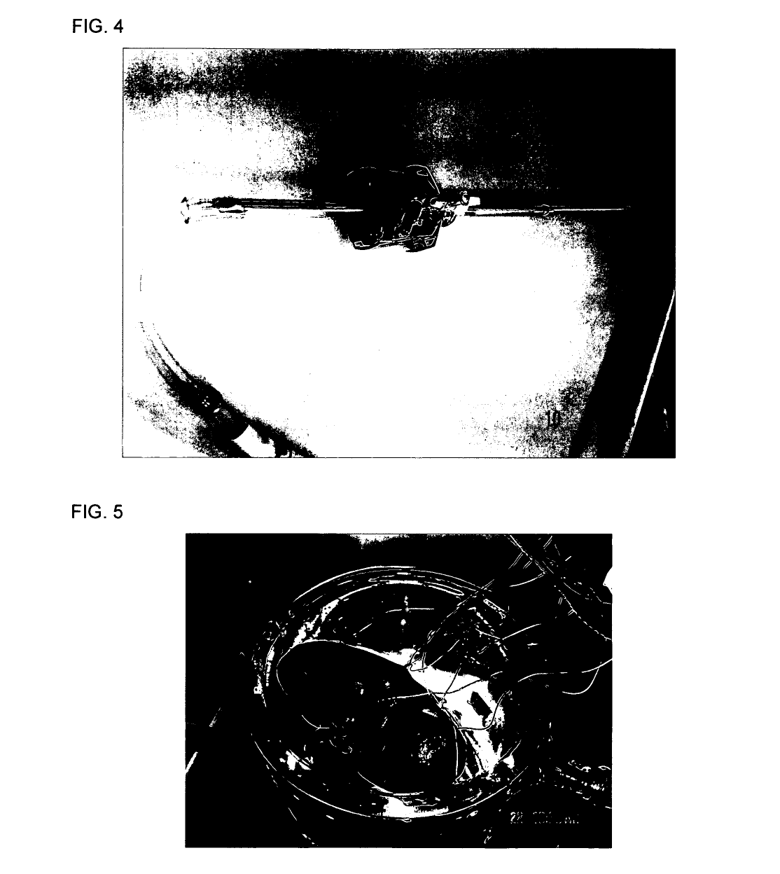

This series of studies presents experimental animal data on cooling intact kidneys in a large swine using our ice slurry pumped and delivered through a specially developed delivery tube through standard laparoscopic ports (FIGS. 2 and 4) guided by endoscope viewing (FIG. 3) to quickly place a coating of slurry over the outside of the kidney. Once the kidney is cooled, the delivery tube is used to remove the slurry melt (e.g., saline) from the cavity surrounding the kidney and/or to partially remove slurry from a region for allowing surgical access, procedures that are likewise guided by endoscope viewing.

The two kidneys of a 42.8 kg swine were used in these experiments. Both kidneys weighted nominally 100 g and were instrumented with 8 fine gauge stainless steel sheathed thermocouples imbedded at various locations 10 mm into tissue to allow tracking of organ temperature during slurry cool-down. For both kidneys, the renal artery and renal vein were clamped off just prior to beginning delivery of the slurry. This clamping procedure is also utilized in human kidney surgery and raises the potential for ischemia-induced damage which, for un-cooled kidney surgery, limits clamp-off time to less than about 30 minutes.

For both kidneys, the ice slurry was made onsite and on demand. For each experiment, the actual ice content after slurry production and immediately prior to delivery was measured using a calorimeter. For both tests, the ice content in % weight of the total slurry mixture was about 40%.

The first kidney of the sedated swine was prepared via laparoscopic transperitoneal access with all kidney preparation, thermocouple placement and slurry delivery guided by endoscopic viewing. Following cooling of the first kidney, the second kidney was prepared via open retroperitoneal access through a flank incision for thermocouple placement and slurry delivery. The procedures used on the second kidney do not constitute minimally invasive surgery and represent conventional open-cavity surgery. However, as this series of experiments clearly demonstrates, slurry cooling has benefits in both minimally invasive and conventional surgery. For both kidney cooling tests, the slurry was delivered using a tubing roller pump (FIG. 7) at a rate of about 200 ml/min. The following describes the two cooling tests performed.

Kidney 1—Laparoscopic Transperitoneal Access

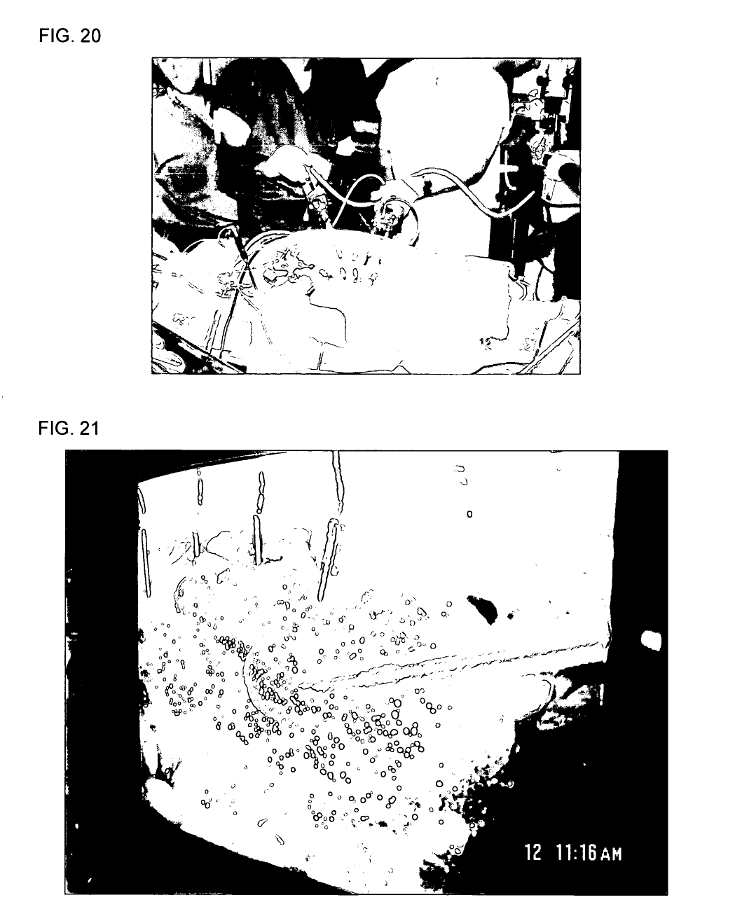

FIGS. 19 and 20, respectively, show the endoscope internal view of the first kidney prior to placement of thermocouples in the kidney, and an external view of the animal showing routing of thermocouples and delivery of slurry during laparoscopic surgery. FIG. 21 shows an endoscope view of the kidney nearly completely covered with slurry as well as the actual SS delivery tip depositing the slurry. FIG. 21 also shows 4 of the 8 thermocouple leads associated with the thermocouples imbedded in the kidney.

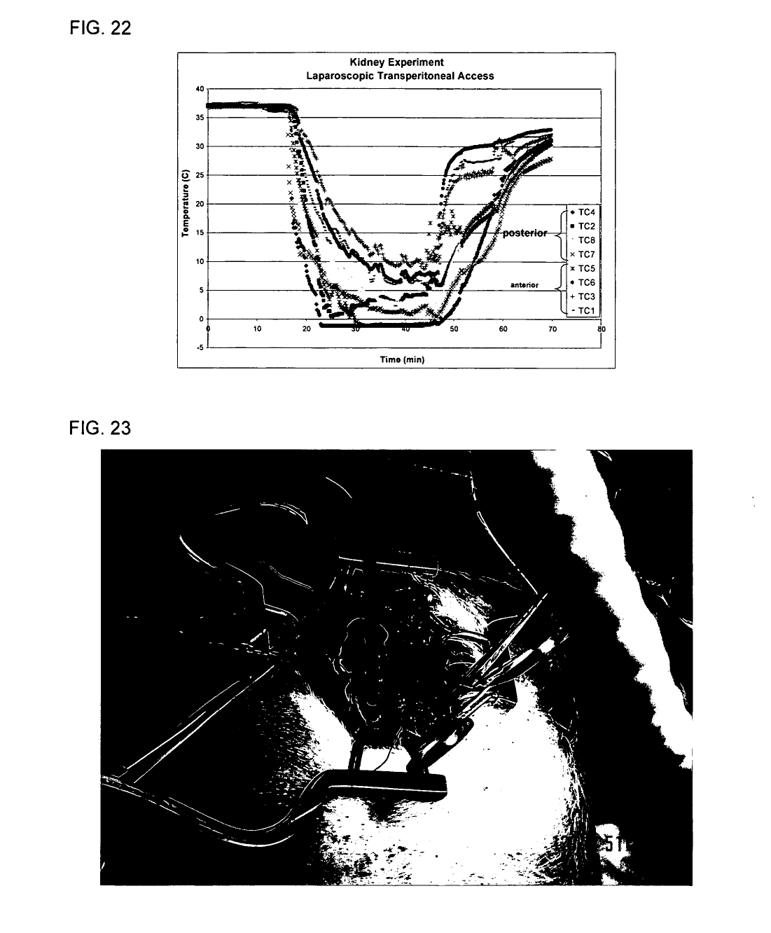

FIG. 22 shows a plot of kidney temperature as a function of time prior to cooling, during delivery of slurry, for a period of time after slurry was delivered, and after unclamping of the blood vessels. As shown in FIG. 22, the targeted cool down of at least 15° C. was achieved quickly in less than 5 minutes, with considerable more cool down occurring than needed indicating that less than the approximately 1 liter of slurry delivered could have been used. The data also indicate that the cool down with one delivery of slurry remains below the target temperature for more than one hour. As further shown in FIG. 22, after much of the slurry has melted and when the kidney blood vessels are unclamped at approximately 47 minutes, the kidney warms up rather quickly and at 70 minutes is nearly back to normal temperature. The warm-up could have been accelerated by removing slurry through a suction tube or by delivering warm saline to melt the slurry surrounding the kidney.

Kidney 2—Open Retroperitoneal Access Through a Flank Incision

As shown in FIG. 23, the second kidney was prepared via open cavity retroperitoneal access through a flank incision for thermocouple placement and slurry delivery. As further shown in FIG. 23, slurry is being pump delivered and deposited in a coating around the kidney by direct visual viewing. Various clamps are used to keep the cavity in which the kidney resides open.

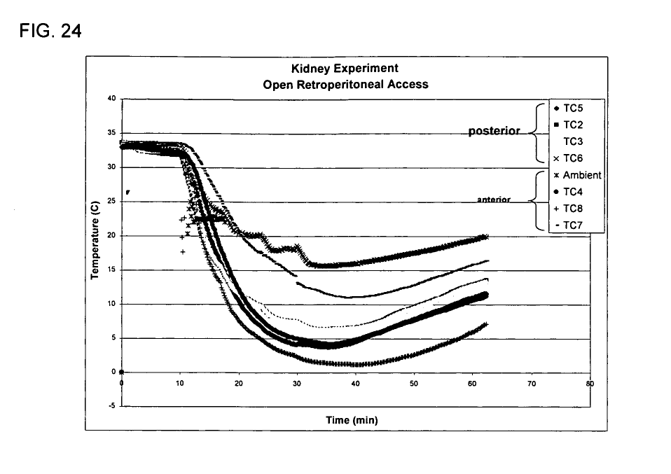

FIG. 24 shows a plot of kidney temperature as function of time prior to cooling, during delivery of slurry, and for a period of time after slurry was delivered. As for the laparoscopic cooling test, the targeted cool down of at least 15° C. was achieved quickly in less than 5 minutes, with considerable more cool down occurring than was needed, indicating that less than the approximately 1 liter of slurry delivered could have been used. The data also indicate that the cool down with one delivery of slurry remains below the target temperature for more than one hour.

Sterility of phase-change particulate slurries used in accordance with the present invention is desirable for most surgical applications. Currently, in human conventional open-cavity kidney surgery, a commercial medical ice maker, which makes dendritic flake-ice, is used to generate ice for hand packing around the kidney. As shown in FIG. 1, this ice is not in the form of a slurry and, due to its dendritic nature, makes a material of very poor fluidity (e.g., the dendritic ice pumps poorly, plugs delivery tubing, and the ice particles become entangled and freeze together).

The level of sterility associated with the currently used open-cavity ice packing technique is readily achievable with the ice slurries used in accordance with the present invention. For example, the ice slurry may be made with medical grade sterile water and salt in a blender which is autoclavable and open to the atmosphere. The slurry is then pumped using a tubing pump (FIG. 7) through a sterile plastic tube into a sterile injector metal delivery tube (FIG. 4).

In conclusion, both of the intact kidney cooling tests described above provide experimental proof of the workability and effectiveness of using ice slurry cooling to prevent ischemia damage during minimally invasive laparoscopic surgery and also during conventional open-cavity surgery.

In addition to the above described laparoscopic transperitoneal kidney cooling tests, survival testing involving 6 animals (swine) were conducted for a clamp-off time of 90 minutes with full external cooling of the kidney to quantify protection from ischemia induced damage. These tests proved that there was no discernible damage and, indeed, that the kidneys were protected by the use of ice slurry.

The foregoing detailed description, representative examples, and accompanying drawings have been provided by way of explanation and illustration, and are not intended to limit the scope of the appended claims. Many variations in the representative embodiments illustrated herein will be apparent to one of ordinary skill in the art, and remain within the scope of the appended claims and their equivalents.

1. A method of inducing protective hypothermia of an organ comprising:
delivering a phase-change particulate slurry to at least a portion of the organ; and
reducing a temperature of the organ through heat exchange with the phase-change particulate slurry, such that a therapeutically protective level of organ hypothermia is induced and a therapeutically acceptable duration of artificially-induced ischemia of the organ is prolonged.

2. The invention of claim 1 further comprising performing a surgical procedure on the organ.

3. The invention of claim 2 wherein the surgical procedure is performed after the temperature of the organ has been reduced.

4. The invention of claim 1 wherein the temperature of the organ is reduced by at least about 10 degrees Celsius.

5. The invention of claim 1 wherein the temperature of the organ is reduced by at least about 15 degrees Celsius.

6. The invention of claim 1 wherein the temperature of the organ is reduced by at least about 20 degrees Celsius.

7. The invention of claim 1 wherein the temperature of the organ is reduced by at least about 25 degrees Celsius.

8. The invention of claim 1 wherein an initial temperature of the organ is about 37 degrees Celsius.

9. The invention of claim 8 wherein the initial temperature is reduced to about 15 degrees Celsius or lower.

10. The invention of claim 8 wherein the initial temperature is reduced to about 15 degrees Celsius or lower in less than about 20 minutes.

11. The invention of claim 8 wherein the initial temperature is reduced to about 15 degrees Celsius or lower in less than about 15 minutes.

12. The invention of claim 8 wherein the initial temperature is reduced to about 15 degrees Celsius or lower in less than about 10 minutes.

13. The invention of claim 2 wherein the surgical procedure is selected from the group consisting of disease diagnosis, disease treatment, and a combination thereof.

14. The invention of claim 2 wherein the surgical procedure is selected from the group consisting of partial organ removal, total organ removal, partial organ replacement, total organ replacement, and combinations thereof.

15. The invention of claim 2 wherein the surgical procedure comprises a minimally invasive procedure.

16. The invention of claim 15 wherein the minimally invasive procedure is selected from the group consisting of laparoscopic procedures, laparoscopically-assisted procedures, thoracoscopic procedures, endoscopic procedures, and combinations thereof.

17. The invention of claim 15 wherein the minimally invasive procedure comprises a laparoscopic technique, and wherein the phase-change particulate slurry is delivered to the organ through an access port in a laparoscope.

18. The invention of claim 2 wherein the surgical procedure comprises an open-cavity procedure.

19. The invention of claim 1 wherein at least a portion of an exterior surface of the organ is coated with the phase-change particulate slurry.

20. The invention of claim 19 further comprising sculpting a coating of the phase-change particulate slurry.

21. The invention of claim 19 further comprising removing at least a portion of the phase-change particulate slurry from the exterior surface of the organ after the temperature of the organ is reduced to a desired value.

22. The invention of claim 1 wherein at least a portion of the phase-change particulate slurry is delivered to an interior surface of the organ.

23. The invention of claim 22 wherein the phase-change particulate slurry is delivered through a biological channel coupled to the interior surface.

24. The invention of claim 23 wherein the biological channel is selected from the group consisting of hila, veins, arteries, and combinations thereof.

25. The invention of claim 22 wherein the organ is at least partially collapsed prior to delivery of the phase-change particulate slurry.

26. The invention of claim 1 wherein at least a portion of the phase-change particulate slurry is delivered to an exterior surface of the organ and wherein at least a portion of the phase-change particulate slurry is delivered to an interior surface of the organ.

27. The invention of claim 1 wherein the phase-change particulate slurry comprises an ice slurry, wherein ice particles are suspended in a carrier comprising water.

28. The invention of claim 27 wherein the ice particles comprise an average diameter of about 100 micrometers or less.

29. The invention of claim 28 wherein the ice particles comprise a substantially globular shape, and wherein the ice particles are substantially smooth.

30. The invention of claim 27 wherein the phase-change particulate slurry further comprises at least one chemical additive.

31. The invention of claim 30 wherein the chemical additive comprises a freezing point depressant.

32. The invention of claim 31 wherein the freezing point depressant comprises sodium chloride.

33. The invention of claim 32 wherein a saline concentration of the phase-change particulate slurry is biocompatible with a host comprising the organ.

34. The invention of claim 33 wherein the saline concentration is between about 0.5 and about 3.0 percent by weight.

35. The invention of claim 27 wherein the ice particles comprise at least about 20 percent by weight of the phase-change particulate slurry.

36. The invention of claim 27 wherein the ice particles comprise at least about 30 percent by weight of the phase-change particulate slurry.

37. The invention of claim 27 wherein the ice particles comprise at least about 40 percent by weight of the phase-change particulate slurry.

38. The invention of claim 27 wherein the ice particles comprise at least about 50 percent by weight of the phase-change particulate slurry.

39. The invention of claim 27 wherein the phase-change particulate slurry is delivered to the organ at a rate of at least about 100 milliliters per minute.

40. The invention of claim 27 wherein the phase-change particulate slurry is delivered to the organ at a rate of at least about 150 milliliters per minute.

41. The invention of claim 27 wherein the phase-change particulate slurry is delivered to the organ at a rate of at least about 175 milliliters per minute.

42. The invention of claim 27 wherein the phase-change particulate slurry comprises sterile ice particles and sterile water.

43. The invention of claim 32 wherein the phase-change particulate slurry comprises sterile ice particles, sterile water, and sterile sodium chloride.

44. The invention of claim 1 further comprising inducing ischemia of the organ.

45. The invention of claim 44 wherein the ischemia is induced via full hilar clamping.

46. The invention of claim 44 wherein the ischemia is induced via partial hilar clamping.

47. The invention of claim 1 wherein the organ is selected from the group consisting of kidneys, liver, pancreas, heart, brain, appendix, spleen, colon, lungs, bladder, prostate, stomach, and combinations thereof.

48. The invention of claim 1 wherein the organ comprises a kidney.

49. The invention of claim 48 further comprising performing a surgical procedure on the kidney.

50. The invention of claim 49 wherein the surgical procedure comprises a full or partial nephrectomy.

51. The invention of claim 50 wherein the nephrectomy comprises a minimally invasive procedure.

52. The invention of claim 50 wherein the nephrectomy comprises an open-cavity procedure.

53. The invention of claim 50 wherein the nephrectomy comprises a laparoscopic technique, and wherein the phase-change particulate slurry is delivered to the kidney through an access port in a laparoscope.

54. The invention of claim 50 wherein at least a portion of an exterior surface of the kidney is coated with the phase-change particulate slurry.

55. The invention of claim 50 wherein at least a portion of the phase-change particulate slurry is delivered to an interior surface of the kidney.

56. The invention of claim 55 wherein the phase-change particulate slurry is delivered through a ureter of the kidney.

57. A method of inducing protective hypothermia of an organ targeted for a surgical procedure comprising:
delivering a phase-change particulate slurry to at least a portion of the organ;
reducing an initial temperature of the organ to about 15 degrees Celsius or lower through heat exchange with the phase-change particulate slurry;
inducing ischemia of the organ; and
performing a surgical procedure on the organ;
wherein the phase-change particulate slurry comprises an ice slurry comprising substantially globular ice particles suspended in a carrier comprising water;
wherein the ice particles comprise an average diameter of about 100 micrometers or less; and
wherein the ice particles comprise at least about 30 percent by weight of the phase-change particulate slurry.

58. The invention of claim 57 wherein the phase-change particulate slurry further comprises sodium chloride.

59. The invention of claim 57 wherein the surgical procedure comprises a minimally invasive procedure.

60. The invention of claim 57 wherein the minimally invasive procedure comprises a laparoscopic technique.

61. The invention of claim 57 wherein the surgical procedure comprises an open-cavity procedure.

62. The invention of claim 57 wherein at least a portion of the phase-change particulate slurry is delivered to an exterior surface of the organ.

63. The invention of claim 57 wherein at least a portion of the phase-change particulate slurry is delivered to an interior surface of the organ.

64. The invention of claim 57 wherein at least a portion of the phase-change particulate slurry is delivered to an exterior surface of the organ and wherein at least a portion of the phase-change particulate slurry is delivered to an interior surface of the organ.

65. The invention of claim 57 wherein all ingredients of the phase-change particulate slurry are substantially sterile.

66. The invention of claim 57 wherein the organ is selected from the group consisting of kidneys, liver, pancreas, heart, brain, appendix, spleen, colon, lungs, bladder, prostate, stomach, and combinations thereof.

67. The invention of claim 57 wherein the organ comprises a kidney.

68. The invention of claim 67 wherein the surgical procedure comprises a full or partial nephrectomy.
